SEQUENCE LISTING (10 sequences)

SEQ ID NO: 1 - protein, 5 aa, synthetic construct
AYSVN

SEQ ID NO: 2 - protein, 16 aa, synthetic construct
MIWGDGKIVYNSALKS

SEQ ID NO: 3 - protein, 10 aa, synthetic construct
DGYYPYAMDN

SEQ ID NO: 4 - protein, 15 aa, synthetic construct
RASKSVDSYGNSFMH

SEQ ID NO: 5 - protein, 7 aa, synthetic construct
LASNLES

SEQ ID NO: 6 - protein, 9 aa, synthetic construct
QQNNEDPRT

SEQ ID NO: 7 - protein, 117 aa, synthetic construct
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 111 aa, synthetic construct [sequence omitted]

SEQ ID NO: 9 - protein, 445 aa, synthetic construct
  VARIANT 1: note = X at position 1 is Q, pE, or absent
  VARIANT 444: note = X at position 444 is G or absent
  VARIANT 445: note = X at position 445 is K or absent
XVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 10 - protein, 218 aa, synthetic construct
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0352644 A1
(43) Pub. Date: Nov. 20, 2025

(54) IL-13 ANTIBODIES FOR THE TREATMENT OF ATOPIC DERMATITIS

(71) Applicant: DERMIRA, INC., Indianapolis, IN (US)

(72) Inventor: Renata Gontijo LIMA, Ponte Vedra, FL (US)

(21) Appl. No.: 18/862,867

(22) PCT Filed: May 3, 2023
(86) PCT No.: PCT/US2023/066527

§ 371 Date: Nov. 4, 2024

Related U.S. Application Data
(60) Provisional application No. 63/338,588, filed on May 5, 2022.

(51) Int. Cl. A61K 39/395; A61K 9/00; A61K 31/573; A61K 39/00; C07K 16/24

(52) U.S. Cl. CPC A61K 39/3955; A61K 9/0014; A61K 31/573; C07K 16/244; A61K 2039/505; A61K 2039/54; A61K 2039/545

(57) ABSTRACT

Provided herein are methods, uses and pharmaceutical compositions of anti-IL-13 antibodies, such as lebrikizumab, for treating atopic dermatitis in pediatric patients. Also provided herein are doses and dosing regimens for the methods and uses of anti-IL-13 antibodies, such as lebrikizumab, for treating atopic dermatitis in pediatric patients.

### FIELD

The present invention relates to methods, uses and pharmaceutical compositions of anti-IL-13 antibodies, such as lebrikizumab, for treating atopic dermatitis in pediatric patients. The present invention also relates to doses and dosing regimens for the methods and uses of anti-IL-13 antibodies, such as lebrikizumab, for treating atopic dermatitis in pediatric patients.

### BACKGROUND

Atopic dermatitis (AD) is a chronic, relapsing, inflammatory skin disease, characterized by erythematous patches with exudates, xerosis, lichenification, and pruritus. It is one of the most common inflammatory skin disorders in the developed world, with a lifetime prevalence of 10% to 20% (Deckers et al., PLOS One 2012; 7: e39803; Weidinger et al., Lancet 2016; 387:1109-22; Weidinger et al., Nature Reviews Disease Primers 2018; 4:1). According to the estimates of the International Study of Asthma and Allergies in Childhood (ISAAC), AD globally affects 15% to 20% of children and 1% to 3% of adults.

AD is more common in children than adults and is frequently first diagnosed in children (Silverberg, Ann Allergy Asthma Immunol. 2019; 123 (2): 144-151). Most cases of AD begin before 5 years of age (85%) and tend to be associated with allergic sensitization (Bieber et al., N Engl J Med. 2008; 358:1483-94; Kim et al., J Am Acad Dermatol. 2016; 75 (4): 681-687.e11; Silverberg and Duran-McKinster, Dermatol Clin. 2017; 35:351-363). The prevalence of AD peaks during childhood (at an estimated 14%), decreased during adolescence (at approximately 8%) and remains stable through adulthood (6% to 8%). AD is a chronic disease. Although AD can remit spontaneously in young children, it is often the first manifestation of a disorder that gives rise to allergic rhinitis, asthma, and food allergies, commonly known as the atopic march (Illi, J Allergy Clin Immunol. 2004; 113:925-31; Spergel, Immunol Allergy Clin North Am 2010; 30:269-80; Davidson, J Allergy Clin Immunol. 2019 Jan. 9. pii: S0091-6749 (19) 30014-4). Children with moderate-to-severe AD have an approximately 50% risk of developing asthma and a 75% risk of developing allergic rhinitis (Thomsen, ISRN Allergy 2014; 2014:1-7).

Interleukin (IL)-13 is a key mediator of T-helper type 2 (Th2) inflammation and signals through a heterodimeric receptor IL-4Rα/IL-13Rα1. Several lines of evidence suggest that IL-13 is a key pathogenetic component in AD. Increased expression of IL-13 has consistently been reported in AD skin (Tsoi L, et al. Journal of Investigative Dermatology. 2019; 139 (7): 1480-1489; Bieber T. Allergy. 2020; 75 (1): 54-62) and some reports suggest a relationship between IL-13 expression and the severity of disease (La Grutta S, et al., Allergy 60:391-5, 2005). Increased IL-13 has also been reported in the serum of AD patients (Novak N, et al., J Invest Dermatol 2002; 119:870-5; WO2016149276), and several studies have reported an increase in IL-13-expressing T cells in the blood of AD patients (Akdis M, et al., J Immunol 1997; 159:4611-9; Aleksza M, et al., Br J Dermatol 2002; 147:1135-41; La Grutta S, et al., Allergy 2005; 60:391-5).

The therapeutic approach to AD consists primarily of trigger avoidance, skin hydration with bathing, the use of moisturizers, and anti-inflammatory therapies such as topical corticosteroids (TCS). In many patients, treatment with TCS provides some symptomatic relief but does not always adequately control the disease. In those patients who have persistent moderate-to-severe disease not responding adequately to TCS, the step-up options include topical calcineurin inhibitors (TCNIs), phototherapy, and immunosuppressive agents such as oral corticosteroids, cyclosporine, azathioprine, methotrexate, and mycophenolate mofetil. These medicines are not available for patients across the globe. Amongst these, cyclosporine is approved for treatment of moderate to severe AD in many European countries and its use is limited to patients aged 16 years and over (for a maximum of 8 weeks [NEORAL®]). Cyclosporine is a potent immunosuppressant that could lead to increased susceptibility to infections and decreased cancer immunosurveillance. Other commonly recognized toxicities of cyclosporine include hypertension and impaired renal and hepatic function.

For childhood AD, the systemic immunosuppressants are often not recommended by current guidelines as they are associated with a substantial risk of side-effects (Chu, Clin Rev Allergy Immunol. 2021; 61 (2): 114-127). In addition to the well described safety concerns, poor patient satisfaction with treatment is reportedly common. Less than one third of patients report satisfaction with their current treatment regimens.

There remains an unmet medical need for safe and effective therapies and treatment regimens for moderate to severe AD in children.

### SUMMARY OF INVENTION

Provided herein are methods, uses and pharmaceutical compositions of anti-IL-13 antibodies, such as lebrikizumab, for treating atopic dermatitis in pediatric patients. Also provided herein are doses and dosing regimens for the methods and uses of anti-IL-13 antibodies, such as lebrikizumab, for treating atopic dermatitis in pediatric patients.

In one aspect, provided herein are methods of treating moderate to severe atopic dermatitis in a patient in need thereof, and such methods comprise administering lebrikizumab to the patient, wherein the patient is 6 months to 12 years of age and has a weight of at least 6 kilogram (kg). In some embodiments, provided herein are methods of treating moderate to severe atopic dermatitis, and such methods comprise: selecting a patient who has moderate to severe atopic dermatitis and is 6 months to 12 years of age and has a weight of at least 6 kg, and administering lebrikizumab to the patient. In some embodiments, the patient is treated for a treatment period of 16 weeks. During the treatment period, lebrikizumab is administered to the patient at a dose and frequency selected based on the patient's weight. In some embodiments, when the patient has a weight of 40 kg or greater, lebrikizumab is administered to the patient at 500 mg at Week 0 and Week 2, and 250 mg once every two weeks from Week 4 to Week 14 (e.g., at Week 4, Week 6, Week 8, Week 10, Week 12, Week 14). In some embodiments, when the patient has a weight of 15 kg to less than 40 kg, lebrikizumab is administered to the patient at 250 mg at Week 0 and 250 mg once every four weeks from Week 2 to Week 14 (e.g., at Week 2, Week 6, Week 10, Week 14). In some embodiments, when the patient has a weight of 6 kg to less than 15 kg, lebrikizumab is administered at 125 mg at Week 0 and 125 mg once every four weeks from Week 2 to Week 14 (e.g., at Week 2, Week 6, Week 10, Week 14).

In another aspect, provided herein are methods of treating moderate to severe atopic dermatitis in a patient in need thereof, and such methods comprise administering lebrikizumab to the patient, wherein the patient is 12 years to 18 years of age and has a weight of less than 40 kg. In some embodiments, provided herein are methods of treating moderate to severe atopic dermatitis, and such methods comprise: selecting a patient who has moderate to severe atopic dermatitis and is 12 years to 18 years of age and has a weight of less than 40 kg, and administering lebrikizumab to the patient. In some embodiments, the patient is treated for a treatment period of 16 weeks. During the treatment period, lebrikizumab is administered to the patient at a dose and frequency selected based on the patient's weight. In some embodiments, when the patient has a weight of 15 kg to less than 40 kg, lebrikizumab is administered to the patient at 250 mg at Week 0 and 250 mg once every four weeks from Week 2 to Week 14 (e.g., at Week 2, Week 6, Week 10, Week 14). In some embodiments, when the patient has a weight of 6 kg to less than 15 kg, lebrikizumab is administered at 125 mg at Week 0 and 125 mg once every four weeks from Week 2 to Week 14 (e.g., at Week 2, Week 6, Week 10, Week 14).

In another aspect, provided herein are methods of treating moderate to severe atopic dermatitis, and such methods comprising: selecting a patient who has moderate to severe atopic dermatitis and a weight of 6 kg to less than 15 kg, and administering lebrikizumab to the patient at 125 mg at Week 0 and 125 mg once every four weeks from Week 2 to Week 14. In some embodiments, the patient is 6 months to 12 years of age. In some embodiments, the patient is 12 years to 18 years of age.

In another aspect, provided herein are methods of treating moderate to severe atopic dermatitis, and such methods comprising: selecting a patient who has moderate to severe atopic dermatitis and a weight of 15 kg to less than 40 kg, and administering lebrikizumab to the patient at 250 mg at Week 0 and 250 mg once every four weeks from Week 2 to Week 14. In some embodiments, the patient is 6 months to 12 years of age. In some embodiments, the patient is 12 years to 18 years of age.

In another aspect, provided herein are methods of treating moderate to severe atopic dermatitis, and such methods comprising: selecting a patient who has moderate to severe atopic dermatitis and a weight of 40 kg or greater, and administering lebrikizumab to the patient at 500 mg at Week 0 and Week 2, and 250 mg once every two weeks from Week 4 to Week 14. In some embodiments, the patient is 6 months to 12 years of age.

In some embodiments, the patient has an Eczema Area and Severity Index (EASI) score of 16 or greater, an Investigator Global Assessment (IGA) score of 3 or greater, and more than 10% of body surface area (BSA) affected by atopic dermatitis, before administration of lebrikizumab. In some embodiments, the patient had inadequate response to topical corticosteroids before administration of lebrikizumab. In some embodiments, the patient has atopic dermatitis for at least 12 months when the patient is 6 years of age or older. In some embodiments, the patient has atopic dermatitis for at least 6 months when the patient is 6 months to less than 6 years of age.

In some embodiments, the methods further comprise determining the EASI score of the patient after the treatment period, e.g., at Week 16. In some embodiments, the EASI score determined after the treatment period, e.g., at Week 16, is reduced by 75% or greater compared to the EASI score determined prior to administration of lebrikizumab. In some embodiments, the EASI score determined after the treatment period, e.g., at Week 16, is reduced by 90% or greater compared to the EASI score determined prior to administration of lebrikizumab.

In some embodiments, the methods further comprise determining the IGA score of the patient after the treatment period, e.g., at Week 16. In some embodiments, the IGA score determined after the treatment period, e.g., at Week 16, is 0 or 1 and the IGA score determined after the treatment period, e.g., at Week 16, is reduced by 2 points or greater compared to the IGA score determined prior to administration of lebrikizumab.

In some embodiments, the methods further comprise determining the Pruritus numeric rating scale (NRS) score of the patient after the treatment period, e.g., at Week 16. In some embodiments, the Pruritus NRS score determined after the treatment period, e.g., at Week 16, is reduced by 4 points or greater compared to the Pruritus NRS score determined prior to administration of lebrikizumab.

In some embodiments, the methods further comprise determining one or more of the following characteristics of the patient at Week 16: (i) percentage of BSA affected by atopic dermatitis; (ii) DLQI, cDLQI, and/or IDLQI; (iii) PRISM; (iv) SCORAD; (v) PROMIS Anxiety, PROMIS Depression, PROMIS Sleep Disturbance, and/or PROMIS Sleep-Related Impairment; (vi) POEM; (vii) EQ-5D-Y and/or EQ-5D-5L; (viii) mSQAAQ; (ix) DFI; (x) WPAI-AD-CG.

In some embodiments, lebrikizumab is administered subcutaneously to the patient. In some embodiments, lebrikizumab is administered to the patient using a subcutaneous administration device, e.g., a prefilled syringe, disposable pen injection device, microneedle device, microinfuser device, needle-free injection device, or autoinjector device.

In another aspect, provided herein are lebrikizumab, or pharmaceutical composition comprising lebrikizumab, for use in treating moderate to severe atopic dermatitis in a patient who is 6 months to 12 years of age and has a weight of at least 6 kg. Also provided are uses of lebrikizumab in the manufacture of a medicament for treating moderate to severe atopic dermatitis in a patient who is 6 months to 12 years of age and has a weight of at least 6 kg.

In another aspect, provided herein are lebrikizumab, or pharmaceutical composition comprising lebrikizumab, for use in treating moderate to severe atopic dermatitis in a patient who is 12 years to 18 years of age and has a weight of less than 40 kg. Also provided are uses of lebrikizumab in the manufacture of a medicament for treating moderate to severe atopic dermatitis in a patient who is 12 years to 18 years of age and has a weight of less than 40 kg.

In another aspect, provided herein are lebrikizumab, or pharmaceutical composition comprising lebrikizumab, for use in treating moderate to severe atopic dermatitis in a patient who has a weight of 6 kg to less than 15 kg. Also provided are uses of lebrikizumab in the manufacture of a medicament for treating moderate to severe atopic dermatitis in a patient who has a weight of 6 kg to less than 15 kg. In some embodiments, lebrikizumab is to be administered to such patient at 125 mg at Week 0 and 125 mg once every four weeks from Week 2 to Week 14. In some embodiments, the patient is 6 months to 12 years of age. In some embodiments, the patient is 12 years to 18 years of age.

In another aspect, provided herein are lebrikizumab, or pharmaceutical composition comprising lebrikizumab, for use in treating moderate to severe atopic dermatitis in a patient who has a weight of 15 kg to less than 40 kg. Also provided are uses of lebrikizumab in the manufacture of a medicament for treating moderate to severe atopic dermatitis in a patient who has a weight of 15 kg to less than 40 kg. In some embodiments, lebrikizumab is to be administered to such patient at 250 mg at Week 0 and 250 mg once every four weeks from Week 2 to Week 14. In some embodiments, the patient is 6 months to 12 years of age. In some embodiments, the patient is 12 years to 18 years of age.

In another aspect, provided herein are lebrikizumab, or pharmaceutical composition comprising lebrikizumab, for use in treating moderate to severe atopic dermatitis in a patient who has a weight of 40 kg or greater. Also provided are uses of lebrikizumab in the manufacture of a medicament for treating moderate to severe atopic dermatitis in a patient who has a weight of 40 kg or greater. In some embodiments, lebrikizumab is to be administered to such patient at 500 mg at Week 0 and Week 2, and 250 mg once every two weeks from Week 4 to Week 14. In some embodiments, the patient is 6 months to 12 years of age.

In some embodiments, the methods, uses, and pharmaceutical compositions described herein further comprise administrating one or more topical corticosteroids to the patient. In some embodiments, the topical corticosteroid is triamcinolone acetonide, hydrocortisone, or a combination of triamcinolone acetonide and hydrocortisone. In some embodiments, the topical corticosteroid is administered concomitantly with lebrikizumab.

### BRIEF DESCRIPTION OF THE DRAWINGS

FIG. 1 is a schematic diagram of the Phase 3 study design described in Example 1 (KGBI). Abbreviations: pts=participants; PK=pharmacokinetics; TCS=topical corticosteroids; V=visit; W=week. Footnotes: ª The post-treatment safety follow-up will occur at Week 26 (or approximately 12 weeks after last dose of study intervention); b Participants who complete the study will be eligible to enroll in a long-term extension study. The “decision” in the schema denotes an interim PK and safety assessment that evaluates the first 30 participants enrolled from Cohort 1 before enrollment of younger participants from Cohort 2.

FIG. 2 is a schematic diagram of the Phase 3 study design described in Example 2 (KGBJ). Abbreviations: V=visit; W=week. Footnotes: ª The post-treatment safety follow-up will occur at Week 62 (or approximately 12 weeks after last dose of lebrikizumab).

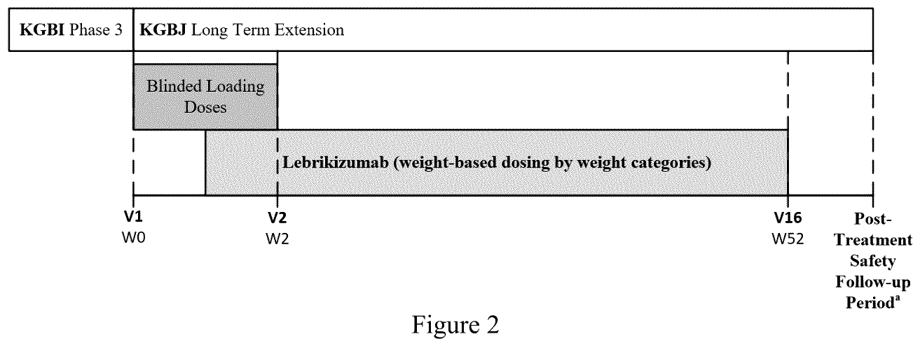

### DETAILED DESCRIPTION

Provided herein are methods, uses and pharmaceutical compositions of anti-IL-13 antibodies, such as lebrikizumab, for treating atopic dermatitis in pediatric patients. Also provided herein are doses and dosing regimens for the methods and uses of anti-IL-13 antibodies, such as lebrikizumab, for treating atopic dermatitis in pediatric patients.

In one aspect, provided herein are methods of treating moderate to severe atopic dermatitis in a patient in need thereof, and such methods comprise administering lebrikizumab to the patient, wherein the patient is 6 months to 12 years of age and has a weight of at least 6 kilogram (kg). In some embodiments, provided herein are methods of treating moderate to severe atopic dermatitis, and such methods comprise: selecting a patient who has moderate to severe atopic dermatitis and is 6 months to 12 years of age and has a weight of at least 6 kg, and administering lebrikizumab to the patient. In some embodiments, the patient is treated for a treatment period of 16 weeks. During the treatment period, lebrikizumab is administered to the patient at a dose and frequency selected based on the patient's weight. In some embodiments, when the patient has a weight of 40 kg or greater, lebrikizumab is administered to the patient at 500 mg at Week 0 and Week 2, and 250 mg once every two weeks from Week 4 to Week 14 (e.g., at Week 4, Week 6, Week 8, Week 10, Week 12, Week 14). In some embodiments, when the patient has a weight of 15 kg to less than 40 kg, lebrikizumab is administered to the patient at 250 mg at Week 0 and 250 mg once every four weeks from Week 2 to Week 14 (e.g., at Week 2, Week 6, Week 10, Week 14). In some embodiments, when the patient has a weight of 6 kg to less than 15 kg, lebrikizumab is administered at 125 mg at Week 0 and 125 mg once every four weeks from Week 2 to Week 14 (e.g., at Week 2, Week 6, Week 10, Week 14).

In another aspect, provided herein are methods of treating moderate to severe atopic dermatitis in a patient in need thereof, and such methods comprise administering lebrikizumab to the patient, wherein the patient is 12 years to 18 years of age and has a weight of less than 40 kg. In some embodiments, provided herein are methods of treating moderate to severe atopic dermatitis, and such methods comprise: selecting a patient who has moderate to severe atopic dermatitis and is 12 years to 18 years of age and has a weight of less than 40 kg, and administering lebrikizumab to the patient. In some embodiments, the patient is treated for a treatment period of 16 weeks. During the treatment period, lebrikizumab is administered to the patient at a dose and frequency selected based on the patient's weight. In some embodiments, when the patient has a weight of 15 kg to less than 40 kg, lebrikizumab is administered to the patient at 250 mg at Week 0 and 250 mg once every four weeks from Week 2 to Week 14 (e.g., at Week 2, Week 6, Week 10, Week 14). In some embodiments, when the patient has a weight of 6 kg to less than 15 kg, lebrikizumab is administered at 125 mg at Week 0 and 125 mg once every four weeks from Week 2 to Week 14 (e.g., at Week 2, Week 6, Week 10, Week 14).

In another aspect, provided herein are methods of treating moderate to severe atopic dermatitis, and such methods comprising: selecting a patient who has moderate to severe atopic dermatitis and a weight of 6 kg to less than 15 kg, and administering lebrikizumab to the patient at 125 mg at Week 0 and 125 mg once every four weeks from Week 2 to Week 14. In some embodiments, the patient is 6 months to 12 years of age. In some embodiments, the patient is 12 years to 18 years of age.

In another aspect, provided herein are methods of treating moderate to severe atopic dermatitis, and such methods comprising: selecting a patient who has moderate to severe atopic dermatitis and a weight of 15 kg to less than 40 kg, and administering lebrikizumab to the patient at 250 mg at Week 0 and 250 mg once every four weeks from Week 2 to Week 14. In some embodiments, the patient is 6 months to 12 years of age. In some embodiments, the patient is 12 years to 18 years of age.

In another aspect, provided herein are methods of treating moderate to severe atopic dermatitis, and such methods comprising: selecting a patient who has moderate to severe atopic dermatitis and a weight of 40 kg or greater, and administering lebrikizumab to the patient at 500 mg at Week 0 and Week 2, and 250 mg once every two weeks from Week 4 to Week 14. In some embodiments, the patient is 6 months to 12 years of age.

In some embodiments, the patient has an Eczema Area and Severity Index (EASI) score of 16 or greater, an Investigator Global Assessment (IGA) score of 3 or greater, and more than 10% of body surface area (BSA) affected by atopic dermatitis, before administration of lebrikizumab. In some embodiments, the patient had inadequate response to topical corticosteroids before administration of lebrikizumab. In some embodiments, the patient has atopic dermatitis for at least 12 months when the patient is 6 years of age or older. In some embodiments, the patient has atopic dermatitis for at least 6 months when the patient is 6 months to less than 6 years of age.

In some embodiments, the moderate to severe atopic dermatitis can be determined by the American Academy of Dermatology Criteria for the Diagnosis and Assessment of Atopic Dermatitis (Eichenfield et al., J Am Acad Dermatol. 2014; 70 (2): 338-351). The essential features that must be present for the diagnosis of AD include pruritus and acute, subacute, or chronic eczema consisting of typical morphology and age-specific patterns, or chronic or relapsing history. Age-specific patterns include facial, neck, and extensor involvement in infants and children; current or prior flexural lesions in any age group; and sparing of the groin and axillary regions. The important features are seen in most cases and support the diagnosis of AD include early age of onset, atopy (personal and/or family history, and/or IgE reactivity), and xerosis. The clinical associations that help to suggest the diagnosis of AD but are too nonspecific to be used for defining or detecting AD for research and epidemiologic studies include atypical vascular responses, such as, facial pallor, white dermographism, and delayed blanch response, keratosis pilaris or pityriasis alba or hyperlinear palms or ichthyosis, ocular or periorbital changes, other regional findings such as, perioral changes or periauricular lesions, and perifollicular accentuation or lichenification or prurigo lesions. A diagnosis of AD also depends on excluding conditions, such as scabies, seborrheic dermatitis, contact dermatitis (irritant or allergic), ichthyoses, cutaneous T-cell lymphoma, psoriasis, photosensitivity dermatoses, immune deficiency diseases, or erythroderma of other causes.

The severity of atopic dermatitis can also be determined by the “Hanifin and Rajka criteria” as described in Hanifin and Rajka diagnostic criteria are described in Acta Derm Venereol (Stockh) 1980; Suppl 92:44-7; or the “Rajka and Langeland criteria,” as described in Rajka G and Langeland T, Acta Derm Venereol (Stockh) 1989; 144 (Suppl): 13-4.

Lebrikizumab is an IgG4 monoclonal antibody that binds human IL-13 with high affinity and blocks IL-13 signaling through the IL-4Rα/IL-13Rα1 heterodimeric receptor. The amino acid sequences of lebrikizumab are provided in Table 1. Lebrikizumab comprises three heavy chain CDRs: HCDR1 of SEQ ID NO: 1, HCDR2 of SEQ ID NO: 2, and HCDR3 of SEQ ID NO: 3, and three light chain CDR3: LCDR1 of SEQ ID NO: 4, LCDR2 of SEQ ID NO: 5, and LCDR3 of SEQ ID NO: 6. Lebrikizumab comprises a heavy chain variable region (VH) of SEQ ID NO: 7 and a light chain variable region (VL) of SEQ ID NO: 8. Lebrikizumab comprises a heavy chain (HC) of SEQ ID NO: 9 and a light chain (LC) of SEQ ID NO: 10. C-terminal clipping of IgG antibodies could occur where one or two C-terminal amino acids are removed from the heavy chain of the IgG antibodies. For example, if a C-terminal lysine (K) is present, it may be truncated or clipped off from the heavy chain. A penultimate glycine (G) may also be truncated or clipped off from the heavy chain as well. Modification of N-terminal amino acid of IgG could also occur. For example, the N-terminal glutamine (Q) or glutamic acid (E) can cyclize into pyro-glutamate (pE) spontaneously. SEQ ID NO: 9 reflects these potential modifications of the lebrikizumab heavy chain.

TABLE 1
Lebrikizumab Sequences

| SEQ ID NO: | Description | Sequence |
|---|---|---|
| 1 | Lebrikizumab | AYSVN |
| | heavy chain | |
| | CDR1 (HCDR1) | |
| 2 | Lebrikizumab | MIWGDGKIVYNSALKS |
| | heavy chain | |
| | CDR2 (HCDR2) | |
| 3 | Lebrikizumab | DGYYPYAMDN |
| | heavy chain | |
| | CDR3 (HCDR3) | |
| 4 | Lebrikizumab | RASKSVDSYGNSFMH |
| | light chain CDR1 | |
| | (LCDR1) | |
| 5 | lebrikizumab | LASNLES |
| | light chain CDR2 | |
| | (LCDR2) | |
| 6 | Lebrikizumab | QQNNEDPRT |
| | light chain CDR3 | |
| | (LCDR3) | |
| 7 | Lebrikizumab | VTLRESGPALVKPTQTLTLTCTVSGFSLSAYSVNWI |
| | heavy chain | RQPPGKALEWLAMIWGDGKIVYNSALKSRLTISKD |
| | variable region | TSKNQVVLTMTNMDPVDTATYYCAGDGYYPYAMD |
| | (VH) | NWGQGSLVTVSS |
| 8 | Lebrikizumab | DIVMTQSPDSLSVSLGERATINCRASKSVDSYGNS |
| | light chain | FMHWYQQKPGQPPKLLIYLASNLESGVPDRFSGS |
| | variable region | GSGTDFTLTISSLQAEDVAVYYCQQNNEDPRTFGG |
| | (VL) | GTKVEIK |
| 9 | Lebrikizumab | Xaa1VTLRESGPALVKPTQTLTLTCTVSGFSLSAYS |
| | heavy chain | VNWIRQPPGKALEWLAMIWGDGKIVYNSALKSRLT |
| | (HC) | ISKDTSKNQVVLTMTNMDPVDTATYYCAGDGYYPY |
| | | AMDNWGQGSLVTVSSASTKGPSVFPLAPCSRSTS |
| | | ESTAALGCLVKDYFPEPVTVSWNSGALTSGVHTFP |
| | | AVLQSSGLYSLSSVVTVPSSSLGTKTYTCNVDHKP |
| | | SNTKVDKRVESKYGPPCPPCPAPEFLGGPSVFLFP |
| | | PKPKDTLMISRTPEVTCVVVDVSQEDPEVQFNWYV |
| | | DGVEVHNAKTKPREEQFNSTYRVVSVLTVLHQDW |
| | | LNGKEYKCKVSNKGLPSSIEKTISKAKGQPREPQV |
| | | YTLPPSQEEMTKNQVSLTCLVKGFYPSDIAVEWES |
| | | NGQPENNYKTTPPVLDSDGSFFLYSRLTVDKSRW |
| | | QEGNVFSCSVMHEALHNHYTQKSLSLSLXaa2Xaa3 |
| | | wherein Xaa1 is Q, pE, or absent; Xaa2 |
| | | is G or absent; Xaa3 is K or absent. |
| 10 | Lebrikizumab | DIVMTQSPDSLSVSLGERATINCRASKSVDSYGNS |
| | light chain (LC) | FMHWYQQKPGQPPKLLIYLASNLESGVPDRFSGS |
| | | GSGTDFTLTISSLQAEDVAVYYCQQNNEDPRTFGG |
| | | GTKVEIKRTVAAPSVFIFPPSDEQLKSGTASVVCLL |
| | | NNFYPREAKVQWKVDNALQSGNSQESVTEQDSK |
| | | DSTYSLSSTLTLSKADYEKHKVYACEVTHQGLSSP |
| | | VTKSFNRGEC |

Lebrikizumab can be formulated with suitable carriers or excipients into a pharmaceutical composition that is suitable for administration to patients. For example, lebrikizumab can be formulated in a pharmaceutical composition as described in WO 2013/066866. The pharmaceutical composition can comprise 125 mg, 250 mg, or 500 mg of lebrikizumab. In some embodiments, lebrikizumab concentration in the pharmaceutical composition is between 100 mg/mL and 150 mg/mL, e.g., 125 mg/mL. The pharmaceutical composition can also comprise 5 mM-40 mM histidine acetate buffer, pH 5.4 to 6.0. In some embodiments, the pharmaceutical composition further comprises a polyol (e.g., sugar) that has a concentration between 100 mM and 200 mM, and/or a surfactant (e.g., polysorbate 20) that has a concentration of 0.01%-0.1%. In one embodiment, the pharmaceutical composition comprises 125 mg/ml of lebrikizumab, 20 mM histidine acetate buffer, pH 5.7, 175 mM sucrose and 0.03% polysorbate 20.

In some embodiments, lebrikizumab is administered subcutaneously to the patient. In some embodiments, lebrikizumab is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). In some embodiments, the subcutaneous administration device is an autoinjector device described in WO 2008/112472, WO 2011/109205, WO 2014/062488, and/or WO 2016/089864.

In some embodiments, the patient is treated with lebrikizumab for a treatment period of 12-68 weeks, e.g., 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks. In some embodiments, the patient is treated with lebrikizumab for a treatment period of 16 weeks. In some embodiments, the patient is treated with lebrikizumab for a treatment period of 52 weeks. In some embodiments, the patient is treated with lebrikizumab for a treatment period of 68 weeks. In some embodiments, the patient is treated with lebrikizumab for a treatment period of 16 weeks, followed by a maintenance period of 52 weeks.

In some embodiments, lebrikizumab is administered to the patient at a loading dose of 250 mg or 500 mg, followed by a subsequent dose of 125 mg or 250 mg for the rest of treatment period. The loading dose can be administered a few times (e.g., once or twice) to the patient at the beginning of the treatment. After the loading dose, lebrikizumab can be administered to the patient at a subsequent dose once every four weeks or once every two weeks. In some embodiments, lebrikizumab is administered at a loading dose of 125 mg at Week 0, followed by a subsequent dose of 125 mg once every four weeks starting from Week 2 for the rest of treatment period. In some embodiments, lebrikizumab is administered at a loading dose of 250 mg at Week 0, followed by a subsequent dose of 250 mg once every four weeks starting from Week 2 for the rest of treatment period. In some embodiments, lebrikizumab is administered at a loading dose of 500 mg at Week 0 and Week 2, followed by a subsequent dose of 250 mg once every two weeks starting from Week 4 for the rest of treatment period.

In some embodiments, lebrikizumab is administered to the patient at a dose and frequency selected based on the patient's weight. In some embodiments, when the patient has a weight of 6 kg to less than 15 kg, lebrikizumab is administered at 125 mg at Week 0 and 125 mg once every four weeks from Week 2 for the rest of treatment period. In some embodiments, when the patient has a weight of 15 kg to less than 40 kg, lebrikizumab is administered to the patient at 250 mg at Week 0 and 250 mg once every four weeks from Week 2 for the rest of treatment period. In some embodiments, when the patient has a weight of 40 kg or greater, lebrikizumab is administered to the patient at 500 mg at Week 0 and Week 2, and 250 mg once every two weeks from Week 4 for the rest of treatment period.

In some embodiments, the treatment period is 16 weeks. In some embodiments, when the patient has a weight of 6 kg to less than 15 kg, lebrikizumab is administered at 125 mg at Week 0 and 125 mg once every four weeks from Week 2 to Week 14 (e.g., at Week 2, Week 6, Week 10, Week 14). In some embodiments, when the patient has a weight of 15 kg to less than 40 kg, lebrikizumab is administered to the patient at 250 mg at Week 0 and 250 mg once every four weeks from Week 2 to Week 14 (e.g., at Week 2, Week 6, Week 10, Week 14). In some embodiments, when the patient has a weight of 40 kg or greater, lebrikizumab is administered to the patient at 500 mg at Week 0 and Week 2, and 250 mg once every two weeks from Week 4 to Week 14 (e.g., at Week 4, Week 6, Week 8, Week 10, Week 12, Week 14).

During and after the treatment period, the patient can be assessed for one or more characteristics of the Atopic Dermatitis Disease Severity Measures (ADDSM), which determine certain signs, symptoms, features, or parameters that have been associated with atopic dermatitis and that can be quantitatively or qualitatively assessed. Exemplary ADDSM include, but are not limited to, Eczema Area and Severity Index (EASI), Investigator Global Assessment (IGA), body surface area (BSA), Severity Scoring of Atopic Dermatitis (SCORAD), Pruritus Numerical Rating Scale (NRS), Parent-Reported Itch Severity Measure (PRISM), Dermatology Life Quality Index (DLQI) score or children's DLQI (CDLQI) or infants' DLQI (IDLQI), modified Subcutaneous Administration Assessment Questionnaire (mSQAAQ), Patient-Reported Outcomes Measurement Information System (PROMIS), PROMIS Anxiety, PROMIS Depression, PROMIS Sleep Disturbance, PROMIS Sleep-Related Impairment, Patient-Oriented Eczema Measure (POEM), European Quality of Life-5 Dimensions-Youth version (EQ-5D-Y), European Quality of Life-5 Dimensions-5 Levels (EQ-5D-5L), Dermatitis Family Impact (DFI), and Work Productivity and Activity Impairment Questionnaire: Atopic Dermatitis Caregiver (WPAI-AD-CG).

The ADDSM can be measured at baseline (prior to the administration of lebrikizumab) and at one or more time points after administration of lebrikizumab. For example, an ADDSM may be measured at the end of Week 1, Week 2, Week 3, Week 4, Week 5, Week 6, Week 7, Week 8, Week 9, Week 10, Week 11, Week 12, Week 13, Week 14, Week 15, Week 16, or longer after the initial treatment with lebrikizumab. The difference between the value of the ADDSM at a particular time point following initiation of treatment and the value of the ADDSM at baseline is used to establish whether there has been an improvement (e.g., a reduction) in the ADDSM.

The “Eczema Area and Severity Index” or “EASI” is an investigator-administered, 20-item scale in adults and children that evaluates 2 dimensions of AD: extent of disease at 4 body regions (head/neck, trunk, upper and lower extremities) and 4 clinical signs (erythema, edema/papulation, excoriation, and lichenification). The clinical signs are assessed for severity on a scale of 0 (absent) to 3 (severe) (Hanifin et al., Exp Dermatol. 2001; 10:11-18). The scores are added up for each of the 4 body regions. The assigned percentages of BSA for each section of the body are 10% for head/neck, 20% for upper extremities, 30% for trunk, and 40% for lower extremities, respectively. Each subtotal score is multiplied by the BSA represented by that region. The multipliers for the region scores are different to reflect the relative proportion of body regions in young children. In addition, an area score of 0 to 6 is assigned for each body region, depending on the percentage of AD-affected skin in that area: 0 (none), 1 (1% to 9%), 2 (10% to 29%), 3 (30% to 49%), 4 (50% to 69%), 5 (70% to 89%), or 6 (90% to 100%). Each of the body area scores are multiplied by the area affected. The resulting EASI ranges from 0 to 72 points, with the highest score indicating worse severity of AD. The recall period of this scale is present time.

The “Investigator Global Assessment” or “IGA” is an is an investigator-administered, single-item scale for rates the severity of the participant's AD (Simpson E, et al. J Am Acad Dermatol. 2020; 83 (3): 839-846). The IGA is composed of a 5-point scale ranging from 0 (clear) to 4 (severe), and a score is selected using descriptors that best describe the overall appearance of the lesions at a given time point (see Table 2).

TABLE 2
Investigator Global Assessment

| Score | Grade | Definition |
|---|---|---|
| 0 | Clear | Minor, residual discoloration; no erythema or |
| | | induration/papulation; no oozing/crusting; no edema. |
| 1 | Almost clear | Trace, faint pink erythema with barely perceptible |
| | | induration/papulation and no oozing/crusting; no edema. |
| 2 | Mild | Faint-pink erythema with papulation and edema |
| | | perceptible upon palpation and no oozing/crusting; |
| | | minimal induration. |
| 3 | Moderate | Pink-red erythema with definite edema of skin papules and |
| | | plaques; there may be some oozing/crusting; palpable |
| | | induration. |
| 4 | Severe | Deep/bright red erythema with significant swelling and |
| | | obvious raised borders of papules and plaques with |
| | | oozing/crusting; significant induration. |

The body surface area (BSA) is an investigator-reported assessment which estimates the extent of disease or skin involvement of a participant's AD. BSA is expressed as a percentage of total body surface and will be reported by body location. BSA is determined by the investigator using the participant's palm is about 1% BSA rule.

The Pruritus Numerical Rating Scale (NRS) is a participant-administered 11-point scale used by participants ≥6 years of age to rate their worst itch severity over the past 24 hours with 0 indicating “No itch” and 10 indicating “Worst itch imaginable.” Assessments are recorded daily by the participant (Yosipovitch et al., Br J Dermatol. 2019; 181 (4): 761-769). Pruritus NRS measures pruritus, which is known best to the participant suffering from AD. As such the question is designed for self-report. Where possible the participant should read and complete the question alone. Where required, a caregiver (parent or other) can read the questions and response options aloud to the person with AD. However, it is important that the participant's selected responses to the questions are entered directly into the question. The caregiver must not influence or question the response given by the person with AD. The Pruritus NRS baseline average score for maximum itch intensity will be determined based on the daily score during the 7 days immediately preceding randomization. A minimum of 4 daily scores out of the 7 days is required to calculate the baseline average score.

The “Parent-Reported Itch Severity Measure” or PRISM is a single-item, parent/caregiver-administered scale that reports the overall severity of their child's itching. Parent/caregiver's report the overall severity of their child's itching based on observed actions of the child in the past 24 hours. Response options range include “No Itch,” “Mild,” “Moderate,” “Severe,” and “Very Severe.” The PRISM is completed for participants <6 years old by the parent/caregiver. A minimum of 4 daily responses out of the 7 days is required to calculate the baseline average response.

The “Severity Scoring of Atopic Dermatitis” or “SCORAD” index is an investigator and participant-administered, 9-item assessment that assesses three aspects: (i) the extent of AD (1-item) is assessed by the investigator as a percentage of each defined body area and reported as the sum of all areas (with the maximum score is 100%); (ii) the severity of 6 specific symptoms of AD: erythema, edema/papulation, oozing/crusts, excoriation, lichenification and dryness (6-items) is assessed by the investigator using a 4-point scale (i.e., none=0, mild=1, moderate=2, severe=3) with a maximum possible total of 18 points; (iii) the subjective symptoms of pruritus and sleep loss (2 items) is assessed by the participant via a 10-cm visual analog scale. The symptoms (itch and sleeplessness) are recorded by the participant or caregiver on a visual analogue scale, where 0 is no symptoms and 10 is the worst imaginable symptom, with a maximum possible score of 20. For participants <8 years, the SCORAD are completed by the caregiver. The SCORAD Index formula is: A/5+7B/2+C. In this formula A is defined as the extent (0-100), B is defined as the severity (0-18), and C is defined as the subjective symptoms (0-20). The maximum possible SCORAD score calculated based on the above 3 aspects is 103. The higher scores indicate poorer or more severe condition (Stalder and Taïeb, Dermatology. 1993; 186 (1): 23-31; Oranje et al., Br J Dermatol. 2007; 157 (4): 645-648; Schram et al., Allergy. 2012; 67 (1): 99-106). The recall period is present time for extent and intensity and average for the last 3 days/nights for subjective symptoms of AD.

The Dermatology Life Quality Index (DLQI) is a participant-administered, 10-item, validated, Quality of Life (QoL) questionnaire in adults that covers 6 domains including symptoms and feelings, daily activities, leisure, work and school, personal relationships, and treatment. The recall period of this scale is over the “last week.” Response categories include “not at all,”, “a little”, “a lot,” and “very much,” with corresponding scores of 0, 1, 2, and 3, respectively, and unanswered (or “not relevant”) responses scored as 0. Scores range from 0 to 30 with higher scores indicating greater impairment of QoL. A DLQI total score of 0 to 1 is considered as having no effect on a participant's health-related Qol (Hongbo et al., J Invest Dermatol. 2005; 125 (4): 659-664), and a 4-point change from baseline is considered as the minimal clinically important difference threshold (Khilji et al. 2002, Br J Dermatol. 2002; 147 (suppl 62): 25-54; Basra et al., Dermatology. 2015; 230 (1): 27-33). The recall period for DLQI is the past week. Participants 4 to <17 years of age will complete the children's DLQI (CDLQI) questionnaire and should continue to complete the cDLQI for the duration of the study (Lewis-Jones M S, Finlay A Y. Br J Dermatol. 1995; 132:942-949). For children below the age of 4 years (that is, up to 3 years 11 months), the caregiver will complete the infants' DLQI (IDQOL) questionnaire and should continue to complete the IDQOL for the duration of the study (Lewis-Jones et al., Br J Dermatol. 2001; 144 (1): 104-110; Basra et al., Br J Dermatol. 2013; 169 (4): 760-768).

The modified Subcutaneous Administration Assessment Questionnaire (mSQAAQ) uses 10 questions that provide an assessment of ease of use and confidence, for example, acceptability and tolerability with using a device to administer a subcutaneous injection of the investigational product. The parents/caregivers of the participants will respond to 10 questionnaire items regarding features related to the use of the administration device using a 7-point Likert scale (from “Strongly Disagree” to “Strongly Agree”) shortly after completing the injection. Features assessed include ease of learning on how to use the device, ease of use, ease of storage of the device, confidence in their ability to use the device, and confidence in the completion of the injection (Callis Duffin et al. 2016). The parent/caregiver completes the questionnaire on paper.

PROMIS (Patient-Reported Outcomes Measurement Information System) is a set of person-centered measures that evaluates and monitors physical, mental, and social health in adults and children (PROMIS WWW). The PROMIS measures include Anxiety and Depression short forms, which assess the participants' symptoms over the previous week. It can be used with the general population and with individuals living with chronic conditions.

The PROMIS Anxiety measures assesses the following items: self-reported fear (fearfulness, panic), anxious misery (worry, dread), hyperarousal (tension, nervousness, restlessness), and somatic symptoms related to arousal (racing heart, dizziness) (PROMIS Anxiety 2019, available at https://www.healthmeasures.net/images/PROMIS/manuals/PROMIS_Anxiety_Scoring_Manual.pdf). The PROMIS Anxiety Short Form (8 questions, 8a v2.0) is available in a pediatric self-report (ages 8 to <18 years) and for parents/caregivers serving as proxy reporters for their children (youth ages ≥5 years). Children aged <5 years will not complete this assessment. Both pediatric self-report and proxy-report versions assess anxiety “in the past 7 days.” Response options range from 1=Never; 2=Rarely; 3=Sometimes; 4=Often; to 5=Almost always. Total raw scores are converted to T-Scores with higher scores representing greater anxiety.

The PROMIS Depression measures assesses the following items: self-reported negative mood (sadness, guilt), views of self (self-criticism, worthlessness), social cognition (loneliness, interpersonal alienation), and decreased positive affect and engagement (loss of interest, meaning, and purpose) (PROMIS Depression 2019, available at https://www.healthmeasures.net/images/PROMIS/manuals/PROMIS_Depression_Scorin g_Manual.pdf). The PROMIS Depression Short Form (8a v2.0 and 6a v2.0) is available in a pediatric self-report (ages 8 to <18 years) and for parents/caregivers serving as proxy reporters for their children (youth ages >5 years). Children aged <5 years will not complete this assessment. Both pediatric self-report and proxy-report versions assess depression “in the past 7 days.” Response options range from 1=Never; 2=Rarely; 3=Sometimes; 4=Often; to 5=Almost always. Total raw scores are converted to T-Scores with higher scores representing greater depression.

The PROMIS Sleep Disturbance instruments assess self-reported perceptions of sleep quality, sleep depth, and restoration associated with sleep. This includes perceived difficulties and concerns with getting to sleep or staying asleep, as well as perceptions of the adequacy of and satisfaction with sleep. The Sleep Disturbance short form is universal rather than disease-specific. It assesses sleep disturbance over the past 7 days. This measurement will be done for locations where translations are available. PROMIS Pediatric Short Form-Sleep Disturbance (8 questions, 8a v1) is available in a pediatric self-report (ages 8 to <18 years) and for parents/caregivers serving as proxy reporters for their children (youth ages ≥5 years). Children aged <5 years will not complete this assessment. Both pediatric self-report and proxy-report versions assess anxiety “in the past 7 days.” Response options range from 1=Never; 2=Rarely; 3=Sometimes; 4=Often; to 5=Almost always. Total raw scores are converted to T-Scores with higher scores representing greater anxiety (Yu L et al., Behav Sleep Med. 2011; 10 (1): 6-24).

The PROMIS Sleep-Related Impairment item banks focus on self-reported perceptions of alertness, sleepiness, and tiredness during usual waking hours, and the perceived functional impairments during wakefulness associated with sleep problems or impaired alertness. Sleep-Related Impairment measures waking alertness, sleepiness, and function within the context of overall sleep-wake function. The Sleep-Related Impairment short form is universal rather than disease-specific. It assesses sleep-related impairment over the past 7 days. This measurement will be done for locations where translations are available. PROMIS Pediatric Short Form-Sleep-Related Impairment (8 questions, 8a v1) is available in a pediatric self-report (ages 8 to <18 years) and for parents/caregivers serving as proxy reporters for their children (youth ages ≥5 years). Children aged <5 years will not complete this assessment. Both pediatric self-report and proxy-report versions assess anxiety “in the past 7 days.” Response options range from 1=Never; 2=Rarely; 3=Sometimes; 4=Often; to 5=Almost always. Total raw scores are converted to T-Scores with higher scores representing greater anxiety (Yu L et al., Behav Sleep Med. 2011; 10 (1): 6-24).

The European Quality of Life-5 Dimensions-Youth version (EQ-5D-Y) is a widely used, generic questionnaire that assesses health status “today” (The EuroQol Group). The EQ-5D-Y is self-completed for pediatric participants >8 years and is completed by parents/caregivers (proxy) for children 4 to <8 years old. This assessment is not completed for children <4 years old per developer recommendation. The questionnaire consists of two parts. The first part assesses 5 dimensions: mobility, self-care, usual activities, pain/discomfort, and anxiety/depression that have 3 possible levels of response (no problems, some problems, or a lot of problems). This part of the EQ-5D-Y can be used to generate a health state index score, which is often used to compute quality-adjusted life year for utilization in health economic analyses. The health state index score is calculated based on the responses to the 3 dimensions, providing a single value on a scale from less than 0 (where zero is a health state equivalent to death; negative values are valued as worse than dead) to 1 (perfect health), with higher scores indicating better health utility. The second part of the questionnaire consists of a visual analog scale on which the participant rates their perceived health state from 0 (“the worst health you can imagine”) to 100 (“the best health you can imagine”). Published studies by EuroQol Group members showed evidence of the instrument's feasibility, reliability, and validity (Ravens-Sieberer et al., Qual Life Res. 2010; 19 (6): 887-897).

The European Quality of Life-5 Dimensions-5 Levels (EQ-5D-5L, EuroQol Research Foundation) is a standardized 5-item self-administered instrument for use as a measure of health outcome. It provides a simple descriptive profile and a single index value for health status that can be used in the clinical and economic evaluation of health care as well as population health surveys. The European Quality of Life-5 Dimensions-5 Levels assesses 5 dimensions of health: mobility, self-care, usual activities, pain/discomfort, and anxiety/depression. The 5L version, scores each dimension at 5 levels: no problems, slight problems, moderate problems, severe problems, and unable to perform/extreme problems. A total of 3125 health states is possible. In addition to the health profile, a single health state index value can be derived based on a formula that attaches weights to each of the levels in each dimension. This index value ranges between less than 0 (where 0 is a health state equivalent to death; negative values are valued as worse than dead) to 1 (perfect health). In addition, the EQ visual analog scale records the respondent's self-rated health status on a vertical graduated (0 to 100) visual analog scale. The participant rates his/her perceived health from 0 (the worst imaginable health) to 100 (the best imaginable health). In conjunction with the health state data, it provides a composite picture of the respondent's health status.

The Patient-Oriented Eczema Measure (POEM) is a caregiver-administered, 7-item, scale that assesses disease severity in children and adults. Participants respond to questions about the frequency of 7 symptoms (itching, sleep disturbance, bleeding, weeping/oozing, cracking, flaking, and dryness/roughness) over the past week. Response categories include “No days,” “1 to 2 days,” “3 to 4 days,” “5 to 6 days,” and “Every day” with corresponding scores of 0, 1, 2, 3, and 4, respectively. Scores range from 0 to 28, with higher total scores indicating greater disease severity (Charman et al., Arch Dermatol. 2004; 140 (12): 1513-1519). A high score is indicative of a poor quality of life. The caregiver is expected to complete all assessments of the POEM even if the participant reaches the age of majority during the course of the study.

The Dermatitis Family Impact (DFI) questionnaire is a caregiver-administered, 10-question, validated, Qol questionnaire that is designed to assess the impact of AD on the Qol of the parents and family members of children with AD (Lawson et al., Br J Dermatol. 1998; 138 (1): 107-113; Dodington et al., Br J Dermatol. 2013; 169 (1): 31-46). The recall period is over the “last week.” Response options include “Not at all,” “A little,” “A lot,” and “Very much,” with corresponding scores of 0, 1, 2, and 3, respectively. Scores range from 0 to 30 with higher scores indicating greater impairment of QoL. The caregiver is expected to complete all assessments of the Dermatitis Family Impact even if the participant reaches the age of majority during the course of the study.

The Work Productivity and Activity Impairment Questionnaire: Atopic Dermatitis Caregiver (WPAI-AD-CG) assesses the effect of a participant's AD on the parent/caregiver's work productivity during the past 7 days. The WPAI-AD-CG consists of 6-items grouped into four domains: absenteeism (work time missed), presenteeism (impairment at work/reduced on-the-job effectiveness), work productivity loss (overall work impairment/absenteeism plus presenteeism), and activity impairment. Scores are calculated as impairment percentages (Reilly et al., Pharmacoeconomics. 1993; 4 (5): 353-365), with higher scores indicating greater impairment and less productivity. The caregiver is expected to complete all assessments of the WPAI-AD-CG even if the participant reaches the age of majority during the course of the study.

In some embodiments, the EASI score of the patient is determined after the treatment period, e.g., at Week 16. In some embodiments, the patient's EASI score determined after the treatment period, e.g., at Week 16, is reduced by 50% or greater compared to the EASI score determined prior to administration of lebrikizumab, which means the patient has achieved “EASI 50”. In some embodiments, the patient's EASI score determined after the treatment period, e.g., at Week 16, is reduced by 75% or greater compared to the EASI score determined prior to administration of lebrikizumab, which means the patient has achieved “EASI 75”. In some embodiments, the patient's EASI score determined after the treatment period, e.g., at Week 16, is reduced by 90% or greater compared to the EASI score determined prior to administration of lebrikizumab, which means the patient has achieved “EASI 90”.

In some embodiments, the IGA score of the patient is determined after the treatment period, e.g., at Week 16. In some embodiments, the patient's IGA score determined after the treatment period, e.g., at Week 16, is 0 or 1 and the IGA score determined after the treatment period, e.g., at Week 16, is reduced by 2 points or greater compared to the IGA score determined prior to administration of lebrikizumab.

In some embodiments, the Pruritus NRS score of the patient is determined after the treatment period, e.g., at Week 16. In some embodiments, the patient's Pruritus NRS score determined after the treatment period, e.g., at Week 16, is reduced by 4 points or greater compared to the Pruritus NRS score determined prior to administration of lebrikizumab.

In some embodiments, one or more of the following characteristics of the patient are determined after the treatment period, e.g., at Week 16: (i) percentage of BSA affected by atopic dermatitis; (ii) DLQI, cDLQI, and/or IDLQI; (iii) PRISM; (iv) SCORAD; (v) PROMIS Anxiety, PROMIS Depression, PROMIS Sleep Disturbance, and/or PROMIS Sleep-Related Impairment; (vi) POEM; (vii) EQ-5D-Y and/or EQ-5D-5L; (viii) mSQAAQ; (ix) DFI; (x) WPAI-AD-CG.

In another aspect, provided herein are lebrikizumab, or pharmaceutical composition comprising lebrikizumab, for use in treating moderate to severe atopic dermatitis in a patient who is 6 months to 12 years of age and has a weight of at least 6 kg. Also provided are uses of lebrikizumab in the manufacture of a medicament for treating moderate to severe atopic dermatitis in a patient who is 6 months to 12 years of age and has a weight of at least 6 kg.

In another aspect, provided herein are lebrikizumab, or pharmaceutical composition comprising lebrikizumab, for use in treating moderate to severe atopic dermatitis in a patient who is 12 years to 18 years of age and has a weigh of less than 40 kg. Also provided are uses of lebrikizumab in the manufacture of a medicament for treating moderate to severe atopic dermatitis in a patient who is 12 years to 18 years of age and has a weigh of less than 40 kg.

In another aspect, provided herein are lebrikizumab, or pharmaceutical composition comprising lebrikizumab, for use in treating moderate to severe atopic dermatitis in a patient who has a weight of 6 kg to less than 15 kg. Also provided are uses of lebrikizumab in the manufacture of a medicament for treating moderate to severe atopic dermatitis in a patient who has a weight of 6 kg to less than 15 kg. In some embodiments, lebrikizumab is to be administered to such patient at 125 mg at Week 0 and 125 mg once every four weeks from Week 2 to Week 14. In some embodiments, the patient is 6 months to 12 years of age. In some embodiments, the patient is 12 years to 18 years of age.

In another aspect, provided herein are lebrikizumab, or pharmaceutical composition comprising lebrikizumab, for use in treating moderate to severe atopic dermatitis in a patient who has a weight of 15 kg to less than 40 kg. Also provided are uses of lebrikizumab in the manufacture of a medicament for treating moderate to severe atopic dermatitis in a patient who has a weight of 15 kg to less than 40 kg. In some embodiments, lebrikizumab is to be administered to such patient at 250 mg at Week 0 and 250 mg once every four weeks from Week 2 to Week 14. In some embodiments, the patient is 6 months to 12 years of age. In some embodiments, the patient is 12 years to 18 years of age.

In another aspect, provided herein are lebrikizumab, or pharmaceutical composition comprising lebrikizumab, for use in treating moderate to severe atopic dermatitis in a patient who has a weight of 40 kg or greater. Also provided are uses of lebrikizumab in the manufacture of a medicament for treating moderate to severe atopic dermatitis in a patient who has a weight of 40 kg or greater. In some embodiments, lebrikizumab is to be administered to such patient at 500 mg at Week 0 and Week 2, and 250 mg once every two weeks from Week 4 to Week 14. In some embodiments, the patient is 6 months to 12 years of age.

In some embodiments, the methods, uses, and pharmaceutical compositions described herein further comprise administrating one or more topical corticosteroids (TCS) to the patient. Exemplary topical corticosteroids include, but are not limited to, triamcinolone acetonide, hydrocortisone, and a combination of triamcinolone acetonide and hydrocortisone. Triamcinolone acetonide is typically formulated at a concentration of 0.1% in a cream, and hydrocortisone is typically formulated at a concentration of 1% or 2.5% in a cream. Certain topical corticosteroids are considered very high potency such as, for example, betamethasone dipropionate, clobetasol propionate, diflorasone diacetate, fluocinonide, and halobetasol propionate. Certain topical corticosteroids are considered high potency such as, for example, amcinonide, desoximetasone, halcinonide, and triamcinolone acetonide. Certain topical corticosteroids are considered medium potency, such as, for example, betamethasone valerate, clocortolone pivalate, fluocinolone acetonide, flurandrenolide, fluocinonide, fluticasone propionate, hydrocortisone butyrate, hydrocortisone valerate, mometasone furoate, and prednicarbate. Certain topical corticosteroids are considered low potency, such as, for example, alclometasone dipropionate, desonide, and hydrocortisone. TCS may be applied to affected areas once daily, twice daily, three times per day, or as needed. TCS use can be recorded using a daily diary. In some embodiments, the patient is inadequately controlled on topical corticosteroids prior to administration of lebrikizumab. In some embodiments, the topical corticosteroid is triamcinolone acetonide, hydrocortisone, or a combination of triamcinolone acetonide and hydrocortisone. In some embodiments, the topical corticosteroid(s) is/are administered concomitantly or sequentially with lebrikizumab. In some embodiments, the topical corticosteroid(s) is/are administered concomitantly with lebrikizumab.

As used herein, the term “a,” “an,” “the” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

The term “flare” as used herein refers to increase in signs and/or symptoms leading to escalation of therapy, which can be an increase in dose, a switch to a higher-potency class of drug, or the start of another drug.

The term “human IL-13” refers to human interleukin 13 (also known as P600), an immunoregulatory cytokine produced primarily by activated Th2 cells. There are two known human IL-13 isoforms: isoform a and isoform b. The term “human IL-13” as used herein refers collectively to all human IL-13 isoforms. The amino acid sequence for human IL-13 isoform a can be found at NCBI Accession No. NP_002179.2. The amino acid sequence for human IL-13 isoform b can be found at NCBI Accession No. NP_001341922.1.

The term “inadequate response” as used herein refers to inability to achieve good disease control of atopic dermatitis (e.g., not able to achieve IGA≤2 or EASI 75) after use of the treatment for the duration recommended by the product prescribing information, or flare of atopic dermatitis occurs while on the treatment.

The term “intolerance” as used herein refers to unacceptable toxicity (e.g., elevated creatinine, elevated liver function tests, uncontrolled hypertension, paranesthesia, headache, nausea, hypertrichosis), or requirement for a drug at doses or duration beyond those specified in the prescribing information.

The term “patient”, as used herein, refers to a human patient.

The term “topical corticosteroid” or “TCS”, as used herein includes Group I, Group II, Group III and Group IV topical corticosteroids. According to the Anatomical Therapeutic Chemical (ATC) Classification System of World Health Organization, the corticosteroids are classified as weak (Group I), moderately potent (Group II) and potent (Group III) and very potent (Group IV), based on their activity as compared to hydrocortisone. Group IV TCS (very potent) are up to 600 times as potent as hydrocortisone and include clobetasol and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### Examples

Example 1. A Randomized, Double-Blind, Placebo-Controlled, Phase 3 Study to Assess the Efficacy, Safety and Pharmacokinetics of Lebrikizumab Compared to Placebo in Participants 6 Months to <18 Years of Age with Moderate-to-Severe Atopic Dermatitis (KGBI)

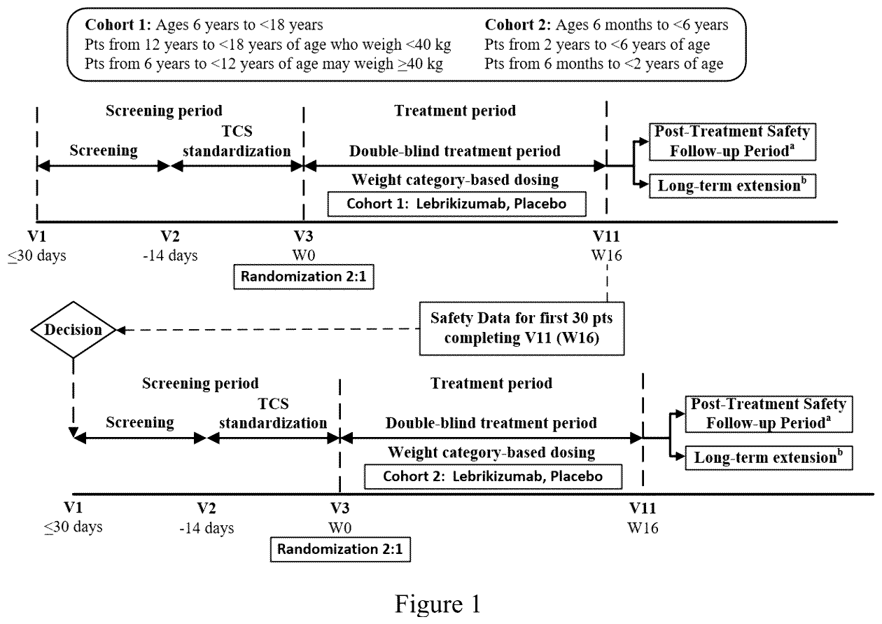

This is a randomized, double-blind, placebo-controlled, Phase 3 study to assess the efficacy, safety and pharmacokinetics of lebrikizumab in participants 6 months to <18 years of age with moderate-to-severe AD. The study design is shown in FIG. 1. Participants are divided into 2 cohorts. Cohort 1 participants are 6 years to <18 years of age, including 12 years to <18 years of age who weigh <40 kg, and 6 years to <12 years of age (who may weigh ≥40 kg). Cohort 2 participants is 6 months to <6 years of age, including 2 years to <6 years of age, and 6 months to <2 years of age.

The study enrollment is staggered by age. Older participants of Cohort 1 are first enrolled into the study. An interim analysis evaluates PK and safety parameters from the first 30 participants completing Week 16 from Cohort 1 (or discontinue early) before enrolling the younger participants of Cohort 2.

### Objectives and Endpoints:

The following primary, secondary, and exploratory objectives and endpoints are evaluated for this study. Statistical analyses of the primary and key secondary endpoints are performed.

TABLE 3
Objectives and Endpoints of KGBI

| | |
|---|---|
| Objectives | Endpoints |

Primary

| | | | |
|---|---|---|---|
| ● | To evaluate the efficacy of lebrikizumab | ● | Percentage of participants with an IGA |
| | when used in combination with TCS | | score 0 or 1 and a reduction ≥2 points from |
| | treatment, compared with placebo, in | | baseline to Week 16, and |
| | participants with moderate-to-severe AD | ● | Percentage of participants achieving EASI- |
| | | | 75, a ≥75% reduction from baseline in |
| | | | EASI score at Week 16 |

Key Secondary

| | | | |
|---|---|---|---|
| ● | To evaluate the efficacy of lebrikizumab | ● | Percentage of participants achieving EASI- |
| | when used in combination with TCS | | 90, a ≥90% reduction from baseline in |
| | treatment, compared with placebo, in | | EASI score at Week 16 |
| | participants with moderate-to-severe AD | ● | Percentage change in EASI score from |
| | | | baseline to Week 16 |
| | | ● | Percentage of participants with a Pruritus |
| | | | NRS of ≥4 points at baseline who achieve |
| | | | a ≥4-point reduction from baseline to Week |
| | | | 16 |
| | | ● | Percentage change in Pruritus NRS score |
| | | | from baseline to Week 16 |
| | | ● | Change in cDLQI from baseline to Week 16 |
| | | ● | Percentage of participants with a Pruritus |
| | | | NRS score of ≥4 points at baseline who |
| | | | achieve both EASI-75 and a ≥4-point |
| | | | reduction in Pruritus NRS score from |
| | | | baseline at Week 16 |

Other Secondary

| | | | |
|---|---|---|---|
| ● | To evaluate the efficacy of lebrikizumab | ● | Mean change from baseline in PRISM at |
| | when used in combination with TCS | | Week 16 |
| | treatment compared with placebo, in | ● | Change in BSA from baseline to Week 16 |
| | participants with moderate-to-severe AD | ● | Percentage change in SCORAD from |
| | | | baseline to Week 16 |
| | | ● | Percentage of participants achieving EASI- |
| | | | 50, a ≥50% reduction from baseline in |
| | | | EASI score at Week 16 |
| ● | To evaluate the pharmacokinetics of | ● | Average serum lebrikizumab concentration |
| | lebrikizumab | | at Week 14 |
| ● | To assess acceptability and tolerability of | ● | As assessed by caregivers using |
| | the administration (caregiver-reported | | mSQAAQ at Week 14 |
| | outcome) | | |

Exploratory

| | | | |
|---|---|---|---|
| ● | Compare the efficacy of lebrikizumab | ● | Proportion of participants with EASI-75, |
| | compared to placebo as measured by | | EASI-90 and EASI-50 by visit |
| | clinician assessments | ● | Percentage change in EASI Score from |
| | | | baseline by visit |
| | | ● | Proportion of participants with IGA Score of |
| | | | 0 or 1 and a reduction ≥2 points from |
| | | | baseline by visit |
| | | ● | Proportion of TCS/TCNI-free days from |
| | | | baseline to Week 16 |
| | | ● | Time (days) to no TCS/TCNI use from |
| | | | baseline to Week 16 |
| ● | Compare the efficacy of lebrikizumab to | ● | Percentage change in Pruritus NRS from |
| | placebo as assessed by patient-reported | | baseline by visit |
| | or caregiver-reported outcomes and | ● | Percentage of participants with a Pruritus |
| | quality of life measures | | NRS score of ≥4 points at baseline who |
| | | | achieve a ≥4-point reduction from baseline |
| | | | by visit |
| | | ● | Change from baseline to Week 16 for these |
| | | | PROMIS measures |

| | |
|---|---|
| ◯ | Anxiety-Pediatric |
| ◯ | Anxiety-Proxy |
| ◯ | Depression-Pediatric |
| ◯ | Depression-Proxy |
| ◯ | Sleep Impairment-Pediatric |
| ◯ | Sleep Impairment-Proxy |
| ◯ | Sleep Disturbance-Pediatric, and |
| ◯ | Sleep Disturbance-Proxy measure |

| | |
|---|---|
| ● | Change from baseline by visit for |

| | |
|---|---|
| ◯ | EQ-5D-5L |
| ◯ | POEM |
| ◯ | DFI Questionnaire |
| ◯ | Work Productivity and Activity |
| | Impairment Questionnaire Atopic |
| | Dermatitis Caregiver |
| ◯ | EQ-5D-Y- Proxy |
| ◯ | EQ-5D-Y- (participants 8 to <16 |
| | years) |
| ◯ | Infant's DLQI (participants <4 years |
| | of age) |
| ◯ | Children's DLQI, and |
| ◯ | DLQI |

| | | | |
|---|---|---|---|
| ● | To evaluate the potential effect of | ● | Changes from baseline in IgE levels |
| | lebrikizumab on immunoglobulin and | ● | Changes from baseline in eosinophil levels |
| | eosinophils | | |

### Study Design:

The study has four treatment periods: Study Periods I to IV. In Study Period I, the appropriate informed consent form and assent form are signed before any study procedures are performed. The investigator will review symptoms, risk factors, medical history, vaccination history concomitant medications and other inclusion and exclusion criteria to confirm eligibility. In Study Period II, a standardized TCS treatment is initiated for all participants and continued through the end of study.

Study Period III is the double-blind treatment period, during which eligible participants are randomly assigned in a 2:1 ratio to receive either lebrikizumab or placebo. Lebrikizumab doses are selected based on the participant's weight (see Table 4).

TABLE 4
Lebrikizumab doses based on the participant's weight.

| Participant Weight | Lebrikizumab Starting Dose(s) | Lebrikizumab Subsequent Doses |
|---|---|---|
| ≥40 kg | Week 0 and Week 2: 500 mg | 250 mg Q2W starting at Week 4 |
| 15 kg to <40 kg | Week 0: 250 mg | 250 mg Q4W starting at Week 2 |
| 6 kg to <15 kg | Week 0: 125 mg | 125 mg Q4W starting at Week 2 |

Q2W = once every 2 weeks; Q4W = once every 4 weeks

Study Period IV is the post-treatment safety follow-up period. For those participants that discontinue the study early or those that complete the study through Week 16 but do not enroll into the long-term extension study, the safety follow-up assessments are conducted 12 weeks after the last dose for final clinical and safety assessment. Participants who complete the study through Week 16 without requiring the use of systemic rescue medication are eligible for enrollment in a long-term extension study with the objective of assessing the long-term safety of lebrikizumab.

### Patient Population:

Approximately 300 participants are enrolled and randomized at a 2:1 ratio to lebrikizumab or placebo (200 participants:100 participants).

Inclusion Criteria: patients eligible for inclusion in this trial must fulfil all of the following criteria:
1. Age and weight requirements: either (1) ≥6 months to <12 years of age and weigh at least 6 kg or (2) ≥12 years of age to <18 years of age and weighing <40 kg.
2. Have a diagnosis of AD prior to screening as stated in the criteria by the American Academy of Dermatology (Eichenfield et al., J Am Acad Dermatol. 2014; 70 (2): 338-351) for at least (1) 12 months if participants are ≥6 years of age, or (2) 6 months if participants are 6 months to <6 years of age.
3. EASI score ≥16 at the screening and baseline visits.
4. IGA score ≥3 (scale of 0 [clear] to 4 [severe]) at the screening and baseline visits.
5. ≥10% body surface area (BSA) of AD involvement at the screening and baseline visits.
6. History of inadequate response to treatment with topical medications. Inadequate response is defined as failure to achieve stable long-term disease control after the use of at least a moderate-potency TCS for at least 4 weeks or for the maximum duration recommended by the product prescribing information (for example, 14 days for super-potent TCS), whichever is shorter, within 6 months of screening.
7. Willing and able to comply with all clinic visits and study-related procedures and questionnaires.
8. For female participants of childbearing potential, remain abstinent or use highly effective contraception.
9. A parent or legal guardian must be able to read, understand, and give documented informed consent for a child to participate in this study.

Exclusion Criteria: participants are excluded from the study if any of the following criteria apply:
1. Are currently enrolled or have participated within the last 8 weeks in a clinical study involving an investigational intervention or any other type of medical research judged not to be scientifically or medically compatible with this study.
2. Treatment with the following prior to the baseline visit:
   a. An investigational drug within 8 weeks or less than 5 half-lives, whichever is longer.
   b. Dupilumab within 8 weeks.
      Note: The enrollment of participants with prior use of dupilumab will be limited to <20%.
   c. B cell-depleting biologics, including rituximab, within 6 months or until lymphocyte and CD 19+lymphocyte count returns to normal, whichever is longer.
   d. Other biologics within 5 half-lives (if known) or 16 weeks, whichever is longer.
3. Have previously been randomized in any other study investigating lebrikizumab.
4. Have experienced hypersensitivity to the active substance or to any of the excipients.
5. Treatment with a topical investigational drug within 2 weeks prior to the baseline visit.
6. Have had an important side effect to TCS, such as, intolerance to treatment, hypersensitivity reactions, significant skin atrophy, and systemic effects, as assessed by the investigator or treating physician that would further preclude use during the study.
7. Treatment with any of the following agents within 4 weeks prior to the baseline visit or any condition that in the opinion of the investigator, is likely to require such treatment(s) during the first 4 weeks of study treatment:
   a. immunosuppressive/immunomodulating drugs (e.g., systemic corticosteroids, cyclosporine, mycophenolate mofetil, IFN-γ, JAK inhibitors, azathioprine, methotrexate).
   b. phototherapy and photochemotherapy for AD.
8. Regular use (more than 2 visits per week) of a tanning booth/parlour within 4 weeks of the screening visit.
9. Use of cannabis or cannabinoids for the treatment of pruritus, pain, and AD.
10. Are currently receiving build-up dosing of allergen immunotherapy (allergy shots).
11. Have received a Bacillus Calmette-Guerin vaccination or treatment within less than 4 weeks before randomization.
12. Have received any live vaccine (that is, live attenuated) within less than 4 weeks before randomization or intend to receive a live vaccine during the study.
13. Have a current or recent acute, active infection requiring treatment with systemic antibiotics, antivirals, antiparasitics, antiprotozoals, or antifungals within 2 weeks prior to screening and until the randomization visit, participants must have no symptoms or signs of confirmed or suspected infection and must have completed any appropriate anti-infective treatment.
14. Have had any of the following types of infection within 3 months of screening or develop any of these infections before baseline:
   a. Serious (requiring hospitalization, and/or intravenous or equivalent oral antibiotic treatment);
   b. Opportunistic (as defined in Winthrop et al., Ann Rheum Dis. 2015; 74 (12): 2107-2116). NOTE: Herpes zoster is considered active and ongoing until all vesicles are dry and crusted over.
   c. Recurring (including, but not limited to recurring cellulitis, or chronic osteomyelitis).
15. Have HIV infection.
16. Have a current or chronic infection with HBV, that is, positive for HBsAg and/or PCR positive for HBV DNA.
17. Have a current infection with HCV, that is, positive for HCV RNA.
18. Have a known active Tuberculosis infection.
19. Have a known active endoparasitic infections or are at high risk of these infections.
20. Have a history or presence of an underlying disease, or surgical, physical, psychological or medical condition that, in the opinion of the investigator would potentially affect participant safety within the study or interfere with the interpretation of data.
21. Have any of the following specific abnormalities on screening laboratory tests:
   a. AST or ALT≥2× upper limit of normal (ULN)
   b. ALP≥2×ULN (Note: Participants may be allowed to enroll if there is no other evidence of liver, bone, or other abnormality but cases must be discussed and judged not clinically significant by medical monitor prior to enrollment.)
   c. total bilirubin ≥1.5×ULN (Note: Participants may be allowed to enroll if there is no other evidence of liver or other abnormality, but cases must be discussed and judged not clinically significant by medical monitor prior to enrollment.)
   d. total white blood cell count <2500 cells/μL (<2.50×10³/μL or <2.50 GI/L)
22. Uncontrolled chronic disease that might require bursts of oral corticosteroids.
23. Uncontrolled asthma as defined by,
   a. Use of systemic steroids (oral or injection) for asthma symptoms in the past 6 months, or
   b. Emergency room, hospital, urgent care, or doctor visits for uncontrolled asthma symptoms (cough, shortness of breath, chest tightness, wheezing) in the past 6 months, or
   c. Use of rescue inhaler or nebulizer more than 2 times a week for asthma symptoms not related to exercise for at least a month (in the past 6 months), or
   d. Waking up during the night more than twice a month due to asthma symptoms (cough, wheezing, shortness of breath)
24. In the opinion of the investigator, have clinically significant laboratory results outside of the reference range at the screening visit.
25. Presence of skin comorbidities that may interfere with study assessments.
26. Have a history of chronic alcohol abuse, intravenous drug abuse, or other illicit drug abuse within the 2 years prior to screening.
27. History of malignancy, including mycosis fungoides, within 5 years before the screening visit. (Exceptions: completely treated in situ carcinoma of the cervix, or completely treated and resolved non-metastatic squamous or basal cell carcinoma of the skin.)
28. Are pregnant or breastfeeding or are planning to become pregnant or breastfeed during the study.
29. Are unsuitable for inclusion in the study in the opinion of the investigator.

Example 2. A Phase 3, Multicenter, Open-Label Extension Study to Assess the Long-Term Safety of Lebrikizumab in Participants 6 Months to <18 Years of Age with Moderate-to-Severe Atopic Dermatitis (KGBJ)

This open-label, Phase 3 study is designed to assess the long-term safety of lebrikizumab in participants 6 months to <18 years of age with moderate-to-severe atopic dermatitis. Participants who have completed study of KGBI (see Example 1) through Week 16 without requiring the use of systemic rescue medication are eligible to enroll into this study KGBJ.

### Objectives and Endpoints:

The following primary, secondary, and exploratory objectives and endpoints are evaluated for this study. Statistical analyses of the primary and key secondary endpoints are performed.

TABLE 5
Objectives and Endpoints of KGBJ

| Objectives | | Endpoints | |
|---|---|---|---|
| Primary | | | |
| ● | To evaluate long-term safety of | ● | Proportion of participants discontinued |
| | lebrikizumab in pediatric participants with | | from study treatment due to adverse |
| | moderate-to-severe AD | | events through the last treatment visit. |
| | | ● | Proportion of participants who reported |
| | | | AEs and SAEs. |
| Secondary | | | |
| ● | To evaluate the efficacy of lebrikizumab in | ● | Percentage of participants with an IGA |
| | pediatric participants with moderate-to- | | score 0 or 1 and a reduction ≥2 points from |
| | severe AD | | baseline of KGBI to Week 52 of KGBJ |
| | | ● | Percentage of participants achieving |
| | | | EASI-75 from baseline of KGBI to Week 52 |
| | | | of KGBJ. |
| | | ● | Change and percentage change from |
| | | | baseline of KGBI to Week 52 of KGBJ in |
| | | | EASI score |
| | | ● | Change in BSA from baseline of KGBI to |
| | | | Week 52 of KGBJ |
| | | ● | Percentage of participants achieving |
| | | | EASI-90 from baseline of KGBI to Week 52 |
| | | | of KGBJ |
| | | ● | Percentage of participants with a Pruritus |
| | | | NRS of ≥4 points at baseline of KGBI who |
| | | | achieve a ≥4 point reduction from baseline |
| | | | of KGBI to Week 52 of KGBJ |
| | | ● | Percentage change from baseline of KGBI |
| | | | to Week 52 of KGBJ for these measures: |

| | |
|---|---|
| ◯ | Pruritus NRS score |
| ◯ | SCORAD |

| ● | To evaluate the pharmacokinetics of | ● | Average serum lebrikizumab |
|---|---|---|---|
| | lebrikizumab | | concentration at Week 50 of KGBJ |
| Exploratory | | | |
| ● | Compare the efficacy of lebrikizumab with | ● | Proportion of participants achieving IGA |
| | that of placebo as assessed by patient- | | score 0 or 1 and a reduction ≥2 points from |
| | reported or caregiver-reported outcomes | | baseline of KGBI by visit |
| | and quality of life measures | ● | Proportion of participants achieving EASI- |
| | | | 75 and EASI-90 from baseline of KGBI by |
| | | | visit |
| | | ● | Change from baseline of KGBI by visit in |
| | | | KGBJ for these measures: |

| | |
|---|---|
| ◯ | PRISM |
| ◯ | DFI |
| ◯ | DLQI |
| ◯ | CDLQI |
| ◯ | IDQOL |
| ◯ | EQ-5D-Y |
| ◯ | EQ-5D-Y- Proxy |
| ◯ | EQ-5D-5L |
| ◯ | POEM |
| ◯ | PROMIS Anxiety-Pediatric |
| ◯ | PROMIS Anxiety-Proxy |
| ◯ | PROMIS Depression-Pediatric |
| ◯ | PROMIS Depression-Proxy |
| ◯ | PROMIS Sleep-Related |
| | Impairment-Pediatric |
| ◯ | PROMIS Sleep-Related |
| | Impairment-Proxy |
| ◯ | PROMIS Sleep Disturbance- |
| | Pediatric |
| ◯ | PROMIS Sleep Disturbance- |
| | Proxy measure |
| ◯ | WPAI-AD-CG |
| ◯ | EASI score |

| | |
|---|---|
| ● | Percentage change from baseline of KGBI |
| | by visit in KGBJ for these measures: |

| | |
|---|---|
| ◯ | Pruritus NRS |
| ◯ | SCORAD |
| ◯ | EASI score |

| | | | |
|---|---|---|---|
| ● | To evaluate the potential effect of | ● | Changes from baseline of KGBI to Week |
| | lebrikizumab on immunoglobulin and | | 52 of KGBJ in IgE levels |
| | eosinophils | ● | Changes from baseline of KGBI to Week |
| | | | 52 of KGBJ in eosinophil levels |

### Study Design:

This is an open-label, Phase 3 study to assess the long-term safety of lebrikizumab in participants 6 months to <18 years of age with moderate-to-severe AD.

Participants who have completed study of KGBI through Week 16 without requiring the use of systemic rescue medication are eligible to enroll into study KGBJ. This study is designed to assess the long-term safety of lebrikizumab for moderate-to-severe atopic dermatitis.

Participants are considered enrolled once all baseline procedures have been completed and the investigator has determined that the participant meets the inclusion and exclusion criteria. The planned duration of treatment for each participant is approximately 52 weeks. All participants will enter a post-treatment follow-up period 12 weeks after the last dose of lebrikizumab.

Table 6 describes the dose and frequency of lebrikizumab used in this clinical study.

TABLE 6
Lebrikizumab doses in KGBJ

| Study | | Study Intervention and Dosage Level(s) in | | |
|---|---|---|---|---|
| Intervention | | Study KGBJ | | |
| Assignment | | Starting Doses at | | |
| from Study | | Week 0 and | | Route of |
| KGBI | Weight | Week 2 | Subsequent Doses | administration |
| Lebrikizumab | 6 kg to <15 kg | Blinding maintained | 125 mg of lebrikizumab | Subcutaneous |
| | | 1 placebo dose at | Q4W starting at Week 6 | injection |
| | | Week 0, and | | |
| | | 1 lebrikizumab | | |
| | | dose at Week 2 | | |
| Lebrikizumab | 15 kg to <40 kg | Blinding maintained | 250 mg of lebrikizumab | |
| | | 1 placebo dose at | Q4W starting at Week 6 | |
| | | Week 0, and | | |
| | | 1 lebrikizumab | | |
| | | dose at Week 2 | | |
| Lebrikizumab | ≥40 kg^{a} | Blinding maintained | 250 mg of lebrikizumab | |
| | | 1 lebrikizumab | Q2W starting at Week 4 | |
| | | dose and 1 placebo | | |
| | | dose at Week 0, | | |
| | | and | | |
| | | 1 lebrikizumab | | |
| | | dose and 1 placebo | | |
| | | dose at Week 2 | | |
| Placebo | 6 kg to <15 kg | 125 mg of | 125 mg of lebrikizumab | |
| | | lebrikizumab | Q4W starting at Week 6 | |
| | | at Weeks 0 and 2 | | |
| Placebo | 15 kg to <40 kg | 250 mg of | 2B50 mg of lebrikizumab | |
| | | lebrikizumab | Q4W starting at Week 6 | |
| | | at Weeks 0 and 2 | | |
| Placebo | ≥40 kg^{a} | 500 mg of | 250 mg of lebrikizumab | |
| | | lebrikizumab | Q2W starting at Week 4 | |
| | | at Weeks 0 and 2 | | |

^{a}Participants who weigh ≥40 kg during the rollover from study KGBI to study KGBJ.

### Patient Population:

Approximately 250 participants will be enrolled to receive lebrikizumab.

Include Criteria: Participants are eligible to be included in the study only if all of the following criteria apply:
1. Received treatment in the study KGBI and have adequately completed the study treatments and last visit of study KGBI.
2. For female participants of childbearing potential, use highly effective contraceptive consistent with local regulations.
3. Are willing and able to comply with all clinic visits and study-related procedures and questionnaires.
4. Provide written informed consent.

Exclusion Criteria: Participants are excluded from the study if any of the following criteria apply:
1. Developed a serious adverse event (SAE) during their participation in study KGBI deemed related to lebrikizumab, which in the opinion of the investigator or of the medical monitor could indicate that continued treatment with lebrikizumab may present an unreasonable risk for the participant*.
2. Developed an adverse event (AE) during their participation in the study KGBI that was deemed related to lebrikizumab and led to study treatment discontinuation, which in the opinion of the investigator or of the medical monitor could indicate that continued treatment with lebrikizumab may present an unreasonable risk for the participant*.
3. Met the protocol-defined criteria for permanent study drug discontinuation in study KGBI, if deemed related to lebrikizumab or led to investigator- or sponsor-initiated withdrawal of participant from the study (e.g., noncompliance, inability to complete study assessments)*.
   * Note for exclusion criteria 1-3: If study KGBI is still blinded at the time of rollover to study KGBJ, conditions deemed related to the study treatment are considered related to lebrikizumab.
4. Are pregnant or breastfeeding or are planning to become pregnant or breastfeed during the study.

The invention claimed is:

1. A method of treating moderate to severe atopic dermatitis in a patient in need thereof, the method comprising administering lebrikizumab to the patient, wherein the patient is 6 months to 12 years of age and has a weight of at least 6 kilogram (kg).

2. A method of treating moderate to severe atopic dermatitis, the method comprising:
selecting a patient who has moderate to severe atopic dermatitis and is 6 months to 12 years of age and has a weight of at least 6 kg, and
administering lebrikizumab to the patient.

3. A method of treating moderate to severe atopic dermatitis in a patient in need thereof, the method comprising administering lebrikizumab to the patient, wherein the patient is 12 years to 18 years of age and has a weight of less than 40 kg.

4. A method of treating moderate to severe atopic dermatitis, the method comprising:
selecting a patient who has moderate to severe atopic dermatitis and is 12 years to 18 years of age and has a weight of less than 40 kg, and
administering lebrikizumab to the patient.

5. The method of claim 1, wherein the patient is treated for a treatment period of 16 weeks.

6. The method of claim 1, wherein when the patient has a weight of 6 kg to less than 15 kg, lebrikizumab is administered to the patient at 125 mg at Week 0 and 125 mg once every four weeks from Week 2 to Week 14.

7. The method of claim 1, wherein when the patient has a weight of 15 kg to less than 40 kg, lebrikizumab is administered to the patient at 250 mg at Week 0 and 250 mg once every four weeks from Week 2 to Week 14.

8. The method of claim 1, wherein when the patient has a weight of 40 kg or greater, lebrikizumab is administered to the patient at 500 mg at Week 0 and Week 2, and 250 mg once every two weeks from Week 4 to Week 14.

9. A method of treating moderate to severe atopic dermatitis, the method comprising:
selecting a patient who has moderate to severe atopic dermatitis and a weight of 6 kg to less than 15 kg, and
administering lebrikizumab to the patient at 125 mg at Week 0 and 125 mg once every four weeks from Week 2 to Week 14.

10. A method of treating moderate to severe atopic dermatitis, the method comprising:
selecting a patient who has moderate to severe atopic dermatitis and a weight of 15 kg to less than 40 kg, and
administering lebrikizumab to the patient at 250 mg at Week 0 and 250 mg once every four weeks from Week 2 to Week 14.

11. A method of treating moderate to severe atopic dermatitis, the method comprising:
selecting a patient who has moderate to severe atopic dermatitis and a weight of 40 kg or greater, and
administering lebrikizumab to the patient at 500 mg at Week 0 and Week 2, and 250 mg once every two weeks from Week 4 to Week 14.

12. The method of claim 9, wherein the patient is 6 months to 12 years of age.

13. The method of claim 9, wherein the patient is 12 years to 18 years of age.

14. The method of claim 1, wherein the patient has an Eczema Area and Severity Index (EASI) score of 16 or greater, an Investigator Global Assessment (IGA) score of 3 or greater, and more than 10% of body surface area (BSA) affected by atopic dermatitis, before administration of lebrikizumab.

15. The method of claim 1, wherein the patient had inadequate response to topical corticosteroids before administration of lebrikizumab.

16. The method of claim 1, wherein the patient has atopic dermatitis for at least 12 months when the patient is 6 years of age or older.

17. The method of claim 1, wherein the patient has atopic dermatitis for at least 6 months when the patient is 6 months to less than 6 years of age.

18. The method of claim 1, wherein lebrikizumab is administered subcutaneously to the patient.

19. The method of claim 1, further comprising determining the EASI score of the patient at Week 16.

20. The method of claim 19, wherein the EASI score determined at Week 16 is reduced by 75% or greater compared to the EASI score determined prior to administration of lebrikizumab.

21. The method of claim 19, wherein the EASI score determined at Week 16 is reduced by 90% or greater compared to the EASI score determined prior to administration of lebrikizumab.

22. The method of claim 1, further comprising determining the IGA score of the patient at Week 16.

23. The method of claim 22, wherein the IGA score determined at Week 16 is 0 or 1 and the IGA score determined at Week 16 is reduced by 2 points or greater compared to the IGA score determined prior to administration of lebrikizumab.

24. The method of claim 1, further comprising determining the Pruritus Numeric Rating Scale (NRS) score of the patient at Week 16.

25. The method of claim 24, wherein the Pruritus NRS score determined at Week 16 is reduced by 4 points or greater compared to the Pruritus NRS score determined prior to administration of lebrikizumab.

26. The method of claim 1, further comprising determining one or more of the following characteristics of the patient at Week 16:
i. Percentage of BSA affected by atopic dermatitis;
ii. DLQI, cDLQI, and/or IDLQI;
iii. PRISM;
iv. SCORAD;
V. PROMIS Anxiety, PROMIS Depression, PROMIS Sleep Disturbance, and/or PROMIS Sleep-Related Impairment;
vi. POEM;
vii. EQ-5D-Y and/or EQ-5D-5L;
viii. mSQAAQ;
ix. DFI;
X. WPAI-AD-CG.

27. The method of claim 1, wherein lebrikizumab is administered to the patient using a subcutaneous administration device.

28. The method of claim 27, wherein the subcutaneous administration device is selected from a prefilled syringe, disposable pen injection device, microneedle device, microinfuser device, needle-free injection device, or autoinjector device.

29. The method of claim 1, wherein the method further comprises administrating one or more topical corticosteroids to the patient.

30. The method of claim 29, wherein the one or more topical corticosteroids is triamcinolone acetonide, hydrocortisone, or a combination of triamcinolone acetonide and hydrocortisone.

31. The method of claim 29, wherein the one or more topical corticosteroids is administered concomitantly with lebrikizumab.

32.-55. (canceled)

56. A pharmaceutical composition comprising lebrikizumab for treating moderate to severe atopic dermatitis in a patient who is 6 months to 12 years of age and has a weight of at least 6 kg.

57.-60. (canceled)
